(19) United States
(12) Patent Application Publication
(10) Pub. No.: US 2008/0305113 A1
(43) Pub. Date: Dec. 11, 2008

(54) Pharmaceutical Composition for Preventing or Treating Chronic Graft-Versus-Disease Comprising Anti-CD137 Monoclonal Antibody

(75) Inventors: Byung Suk Kwon, Nam-gu (KR); Ju Yang Kim, Nam-gu (KR); Hong Rae Cho, Dong-gu (KR)

Correspondence Address: GREENLEE WINNER AND SULLIVAN P C, 4875 PEARL EAST CIRCLE, SUITE 200, BOULDER, CO, 80301, US

(73) Assignee: University of Ulsan Foundation for Industry Cooperation, Nam-gu (KR)

(21) Appl. No.: 12/048,456

(22) Filed: Mar. 14, 2008

(30) Foreign Application Priority Data
Jun. 5, 2007 (KR) 10-2007-0054857

(51) Int. Cl. A61K 39/395; A61P 43/00

(52) U.S. Cl. 424/141.1

(57) ABSTRACT

Provided is a pharmaceutical composition for preventing and treating chronic graft-versus-host disease (cGVHD) containing an anti-CD137 monoclonal antibody.

The pharmaceutical composition containing the anti-CD137 monoclonal antibody as an active component reduces a cytokine produced from a CD4+ T cell, and increases a death of a donor CD4+ T cell, thereby effectively preventing and treating cGVHD, and thus can be useful for allogeneic stem cell transplantation.

### BACKGROUND OF THE INVENTION

1. Field of the Invention

The present invention relates to a pharmaceutical composition for preventing and treating chronic graft-versus-host disease containing an anti-CD137 monoclonal antibody.

2. Description of the Related Art

Chronic graft-versus-host disease (cGVHD) commonly develops in various symptoms in allogeneic stem cell transplant recipients, and is often accompanied by other clinical complications such as diseases like fibrosis and scleroderma (Gilliam A C, J. Invest., Dermatol., 123, 251-257, 2003). It is known that the cGVHD is mediated by a pathogenic donor T cell produced after alloreactivity to a minor histocompatibility (mH) antigen or autoantigen against a host, wherein the T cell attacks a target tissue by stimulating the secretion of infectious and fibrous cytokines, or production of autoantibodies, in addition to direct cytolytic attack (Lee S J, Blood, 105, 4200-4206, 2005). However, except for the use of systemic corticosteroids, little is known about therapeutic treatments for the cGVHD.

The cGVHD is developed by a donor CD4+ T cell. Interestingly, both a donor cell and an antigen-presenting cell (APC) against a host mediate CD4+ T cell-mediated skin GVHD, which is a principal characteristic of the cGVHD, and implies that mH antigens of endogenic and exogenic hosts may be delivered to the donor CD4+ T cells in a major histocompatibility complex (MHC) class II (Anderson B E et al., Blood, 105, 2227-2234, 2005). Further, as it has been disclosed from B10.D2→Balb/c model that immunodominant antigens are expressed in skin and a microenvironment of the skin is good for development of a helper type II CD4+ T cell (Th2 cell) (Kaplan D H et al., J. Immunol., 173, 5467-5475, 2004), it has been discovered that the cGVHD is specifically mediated by the Th2 cell in a H-2^{d} genetic background (Abraham D J et al., Trends Immunol., 26, 587-595, 2005), and developed by cytokines released from the Th2 cells and/or autoantigens (Zhang C. et al., Blood, 107, 2993-3001, 2006).

CD137 belongs to the TNF receptor super family, and serves as a strong costimulatory molecule with respect to a CD8+ T cell (Shuford W W et al., J. Exp. Med., 186, 47-55, 1997; and Takahashi C. et al., J. Immunol., 162, 5037-5040, 1999). Mittler et al. proved that binding of CD137 in vivo inhibits a T cell-dependent antibody reaction, which indicates that an agonistic anti-CD137 monoclonal antibody (mAb) will be helpful for the treatment of the Th2-mediated immune diseases. In practice, except for research on autoimmune cerebrospinal meningitis and autoimmune uveitis, it has been reported that the anti-CD137 mAb is effective in preventing and treating the autoimmune diseases and other immune diseases which are known to be mediated by the autoantibody and the Th2 cell, for example, systemic lupus erythematosus (SLE) (Sun Y. et al., Nat. Med., 8, 1405-1413, 2002; Foell J. et al., Clin. Invest., 111, 1505-1518, 2003), Rheumatoid arthritis (Seo S K et al., Nat. Med., 10, 1088-1094, 2004; and Foell J L et al., Immunology, 113, 89-98, 2004), allergic conjunctivitis (Fukushima A. et al., J. Immunol., 175, 4897-4903, 2005) and allergic asthma (Cho Y S et al., Clin. Exp. Allergy, 36, 377-385, 2006).

Meanwhile, a therapeutic method for IgE-mediated diseases such as asthma, atopy and allergy by administering an anti-CD137 mAb to a mammal is disclosed in U.S. Patent Application No. 20030223989, and a therapeutic method for cancer by neutralization of CD137 or inhibition of CD137 expression is disclosed in U.S. Patent Application No. 2006012103. However, not much is known about the activities of the anti-CD137 mAb for preventing and treating cGVHD.

As a result of research on how to prevent and treat cGVHD which had not been established, the present inventors discovered that a composition containing an anti-CD137 mAb is effective in preventing and treating cGVHD in a B10.D2→Balb/c(H-2^{d}) mH antigen-incompatible model of GVHD. Thus, the present invention is completed.

### SUMMARY OF THE INVENTION

The present invention is directed to a pharmaceutical composition for preventing or treating chronic graft-versus-host disease (cGVHD) containing an anti-CD137 monoclonal antibody.

One aspect of the present invention provides a pharmaceutical composition for preventing or treating cGVHD containing an anti-CD137 monoclonal antibody as an active component.

### BRIEF DESCRIPTION OF THE DRAWINGS

The above and other objects, features and advantages of the present invention will be described in reference to certain exemplary embodiments thereof with reference to the attached drawings in which:

FIG. 1 illustrates a development progress of cGVHD ((i) of A) and a change of weight ((ii) of A) from the first day to the 120^{th} day of antibody administration, and a range of pressure ulcer (B), a mouse's state (C) and a state of a tissue of cGVHD lesion (D) on the 24^{th} day (the 58^{th} day of cell transplantation) of the antibody administration, after an anti-CD137 antibody or Ig as a control is administered to a B10.D2→Balb/c(H-2^{d}) cGVHD mouse model on the 34^{th} day of the cell transplantation;

FIG. 2 illustrates a development progress of cGVHD (A) and a change of weight (B) from the first day to the 54^{th} day of the antibody administration (the 110^{th} day of cell transplantation), and a range of pressure ulcer (C) and a mouse's state (D) on the 54^{th} day of the antibody administration, after an anti-CD137 antibody or Ig as a control is administered to a B10.D2→Balb/c(H-2^{d}) cGVHD mouse model on the 56^{th} day of the cell transplantation; and

FIG. 3 illustrates viability (A), a development progress of cGVHD (B), a range of pressure ulcers (C), a mouse's state (D), and a state of a tissue of cGVHD lesion (E) from the 30^{th} day of anti-CD137 antibody administration or Ig, as a control, treatment to the 30^{th} day (the 60^{th} day of cell transplantation) of the antibody administration, after cells are transplanted to the B10.D2→Balb/c(H-2^{d}) cGVHD mouse model which is induced by a CD4+ T cell and a CD8+ T cell.

### DETAILED DESCRIPTION OF THE INVENTION

The present invention will now be described more fully hereinafter with reference to the accompanying exemplary embodiments and experimental examples. This invention may, however, be embodied in different forms and should not be construed as limited to the embodiments and examples set forth herein.

The present invention provides a composition, which contains an anti-CD137 monoclonal antibody having an agonistic effect by specifically recognizing CD137 and binding thereto. The anti-CD137 monoclonal antibody may use proteins commercially available, or produced or isolated from mammals other than humans. In one embodiment of the present invention, such an anti-CD137 monoclonal antibody was provided from Dr. Mittler of Emory University.

For example, the anti-CD137 monoclonal antibody, an active component of the present composition, may be produced by: immunizing an animal using CD137 as an immunogen, fusing a spleen cell of the immunized animal with a myeloma cell to yield a hybridoma, selecting a positive clone producing a monoclonal antibody specifically recognizing CD137, and culturing the selected hybridoma and then isolating an antibody from culture media for the hydridoma. Further, the monoclonal antibody of the present invention may be produced by inserting the hybridoma into an abdominal cavity of the animal and then isolating it from an ascites thereof in a predetermined period of time. Regarding monoclonal antibodies and hybridomas, the following literature may be referred to [Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988)].

The immunogen may use wild-type CD137 protein or recombinant CD137 protein. The recombinant CD137 may be acquired by ligasing cDNA using a disclosed base sequence through a common method well known in the art, inserting the ligased fragment into an expression vector for gene expression in a host cell, and purifying the CD137 protein.

To obtain an anti-CD137 monoclonal antibody having activities of specifically recognizing the CD137 and inhibiting the activation thereof, an animal is immunized with the immunogen as an antigen. The animal is preferably a mouse or rat. The antigen is administered by a common method such as abdominal, intramuscular, intraocular or subcutaneous injection. If necessary, using various techniques, immune reactions caused by proteins may increase, and higher antibody reactivity may be developed. For example, the immunity of the animal may be improved using the antigen protein in combination with a complete or incomplete supplement (Freund). It is not particularly limited to an immunizing period, but the antigens may be administered two to ten times, and preferably two to five times in intervals of several days to several weeks, and preferably of one to three weeks. An antibody producing cell may be yielded from the animal on the 1^{st} to 10^{th} day, and preferably the 2^{nd} to 5^{th} day of the final immunizing period. The antibody-producing cells include spleen cells, lymphatic cells, thymocytes and peripheral blood cells, and preferably spleen cells. The antigen is administered at a dose of 0.01 to 1000 μg, preferably 1 to 300 μg per mouse.

The antibody-producing cell is fused with the myeloma cell by a well known method, for example, the Koehler & Milstein method. The myeloma cell may include mouse-derived p3/x63-Ag8, p3-U1, NS-1, MPC-11, SP-2/0, F0, p3x63 Ag8. V653 and S194. Further, a rat-derived R-210 cell line may be used. Clones are cultured, selecting therefrom a monoclone specifically recognizing CD137 by an enzyme-linked immunosorbent assay. The selection of the monoclone specifically recognizing CD137 may be performed by any immunochemical methods which are well known in the art, which include but are not limited to the following methods: a radio immunoassay (RIA), an enzyme-linked immunosorbent assay (ELISA), an immunofluorescence assay, Western blotting and a facial action coding system (FACS).

Further, the anti-CD137 monoclonal antibody may be produced by a recombination technique of isolating DNA coding an antibody. Using the isolated DNA, an antibody having an agonistic activity may be produced by a common method of binding to the CD137 with a high affinity. For example, a chain shuffling technique which has been used to produce an antibody having a high affinity to heptene-2-phenylozasol-5-one may be useful (Marks et al., BioTechnology, 10:779, 1992; Schier et al., J. Mol. Biol, 263:551, 1996).

The present inventors defined that the anti-CD137 monoclonal antibody may prevent and treat cGVHD which may develop during blood transfusion or bone marrow transplantation, by increasing the death of a donor Th2 CD4+ T cell in vivo using an animal model specially developed for studying cGVHD.

It has been reported that the animal model developed for studying cGVHD, i.e., the B10.D2→Balb/c(H-2d) mH antigen-incompatible model of GVHD has symptoms shown at the last stage of human cGVHD, for example, dermatofibrosis, ulcer and alopecia accompanied with collagen deposition (Jaffee B D et al., Cell Immunol., 77, 1-12, 1983; McCormick L L et al., J.Immunol., 163, 5693-5699, 1999; Zhang Y et al., J. Immunol., 168, 3088-3099, 2002; and Nonomura A et al., Pathol. Int., 48, 421-427, 1998). Further, such a rat model has histological characteristics such as lichenoid subepithelial infiltrates, follicular drop-out, a decrease in subcutaneous fat and dermal mononuclear infiltrates, pulmonary fibrosis, inflammation and damage of salivary and lachrymal glands, and liver diseases characterized by fibrous thickening and hardening of a wall of a bile duct after mononuclear infiltration of the bile ducts in and out of the liver (Jaffee B D et al., Cell Immunol., 77, 1-12, 1983; Anderson B E et al., J. Clin. Invest., 112, 101-108, 2003: and Li J et al., Transplantation, 62, 1621-1628, 1996).

In one embodiment of the present invention, it is determined from the B10.D2→Balb/c(H-2d) mH antigen-incompatible mouse model of cGVHD whether the anti-CD137 monoclonal antibody has a therapeutic activity of cGVHD by measurement of weight, evaluation of cGVHD signs (clinical score), estimation of the number of mice having ulcer in percent and histological analysis in an early stage of cGVHD induction and after development of cGVHD (see Exemplary Embodiments 1 and 2).

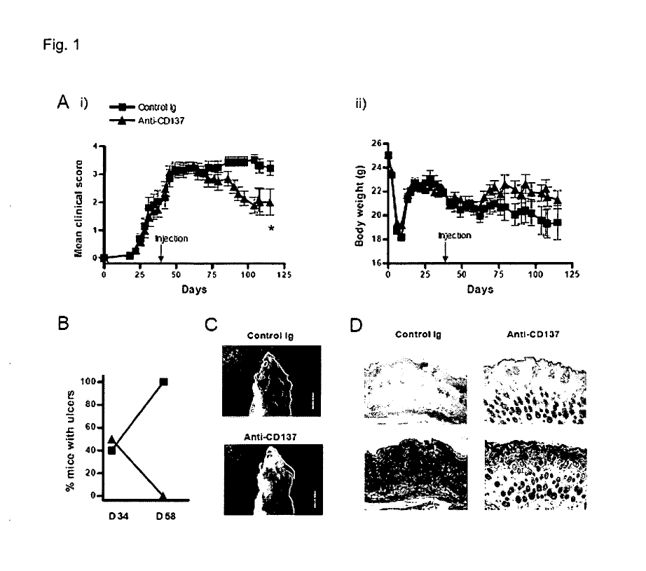

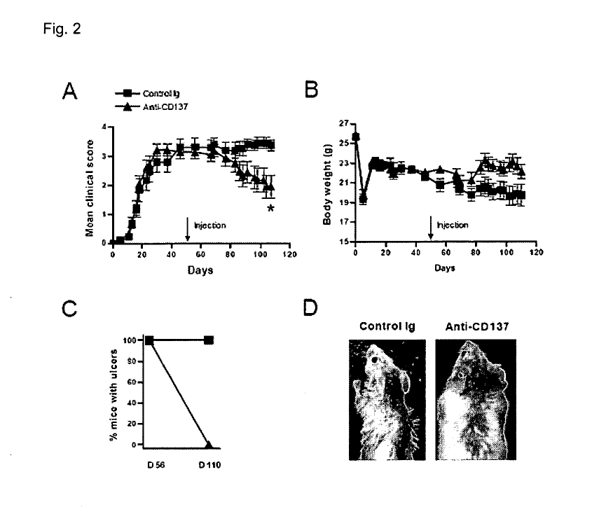

As a result, it may be confirmed that, from the development sign evaluation, the mouse recovers by the administration of the anti-CD137 monoclonal antibody, it is observed that the weight increases, pressure ulcer is treated, and hairs grow from the lesion of the skin, and it may be also confirmed from the histological analysis that the lesions of cGVHD are recovered (see FIGS. 1 and 2). Consequently, it may be noted that the cGVHD can be treated using the anti-CD137 antibody.

It is assumed that the therapeutic activity of the anti-CD137 monoclonal antibody to cGVHD is shown in a following mechanism. When the anti-CD137 monoclonal antibody is administered in the early stage of the GVHD induction, the cell death of a donor CD4+ T cell is induced, and a Th1 cell is activated along with ‘cytokine storm’ associated with acute GVHD (aGVHD) at the same time, thereby developing aGVHD. However, when the anti-CD137 monoclonal antibody is administered after cGVHD has progressed, the cell death of the donor CD4+ T cell is induced by Fas/Fas ligand (FasL) binding, thereby blocking the progression of the cGVDH, and having an excellent therapeutic activity.

Particularly, when the anti-CD137 antibody is administered to the mouse model between the 30^{th} to 60^{th} day of the cGVHD development, the antibody may exhibit the most excellent therapeutic activity.

Thus, the pharmaceutical composition including an anti-CD137 monoclonal antibody according to the present invention is effective in prevention and treatment of cGVHD. Here, the cGVHD may be caused by blood transfusion and transplantation, and the cell transplantation may include bone marrow, stem cell and organ transplantations.

Further, a fragment of the anti-CD137 monoclonal antibody may be used as an active component of the pharmaceutical composition of the present invention. The term “antibody fragment” is referred to as a part of the antibody which may bind to a target antigen, i.e., CD137 or a specific region thereof. For example, the antibody fragments include F(ab′)2, Fab, Fab′ and Fv fragments. These may be generally formed by the recombinant DNA technique, or a conventional method of degrading antibody protein with papain or pepsin (Current Protocols in Immunology, John Wiley and Sons Coliganet al., eds. (1991-92)).

The pharmaceutical composition of the present invention may be administered alone, or in conjugation with other therapeutic drugs, compounds or pharmaceutical compositions by a common method such as injection or infusion by time. For example, though the present invention is not limited hereto, the injection may include oral, intravenous, abdominal, intramuscular, luminal, subcutaneous or percutaneous injection.

When the composition of the present invention is administered orally, the anti-CD137 monoclonal antibody, the active component of the composition, may be formulated in a tablet or capsule in combination with a carrier and a diluent, and when administered non-orally, the anti-CD137 monoclonal antibody may be enclosed in a vial or ample to be administered by intravascular infusion or intramuscular injection after being dissolved in 10% glucose solution, saline, distilled water or a similar liquid. Also, to enhance stability, the composition enclosed in a vial or ample may be lyophilized.

The pharmaceutical composition of the present invention may additionally include at least one kind of pharmaceutically available carriers or diluents, in addition to the anti-CD137 monoclonal antibody, which is the active component, wherein the pharmaceutically available carriers or diluents include at least one of a filler, a binder, a capsulation material, a lubricant, a wetting agent, a disintegrant, an emulsion, a suspension, a sweetening agent and a flavoring agent, each of which is formed in a solid or liquid, and suitable for administration to the mammals including human. Also, the composition of the present invention may further include pharmaceutically permeable amounts of salts, fillers (e.g., acetate, citrate, borate and phosphate), preservatives (e.g., benzalkonium chloride, chlorobutanol, paraben and thimerosal), immunity stimulating agent (e.g., adjuvant and cytokine) or other therapeutic agents (e.g., chemotherapeutic agents). Also, the composition of the present invention may be formulated to rapidly, continuously or naturally release the active component after the administration to a patient.

In the pharmaceutical composition of the present invention, the anti-CD137 monoclonal antibody may be administered at a dose of 5 to 10 mg/kg per day, which may be divided into one or several injections. However, it may be understood that an actual dose of the active component may be determined by various related factors such as an administration route, age, sex, weight and severity of the disease, and thus the scope of the present invention is not limited to the dose described above.

Hereinafter, a particular method of the present invention will be fully described with reference to exemplary embodiments, but the scope of the present invention is not be limited to these embodiments.

### Exemplary Embodiment 1

Therapeutic Effect (1) of Anti-CD137 Antibody in Developing cGVHD

To confirm a therapeutic effect of an anti-CD137 antibody in developing cGVHD, a following experiment was performed using a B10.D2→Balb/c(H-2^{d}) mH antigen-incompatible model. After applying a radiation of 750 cGy to a 6 to 8 week-old Balb/c mouse (Orient, Korea) using a cesium radiation irradiator, 5×10⁶ number of T cell-exhausted bone marrow (BM) cells obtained from a 6 to 8 week-old male B10.D2 mouse (SLC, Japan) only or together with 1×10⁷ number of CD4+ T cells isolated from the B10.D2 mouse were transplanted, thereby obtaining a B10.D2→Balb/c(H-2^{d}) cGVHD model. The mouse model was measured in weight every three days, and the population of mice having GVHD (%), the development sign on skin (clinical score) and their weight were measured on the 15^{th} day of the transplantation. The evaluation standards of the development sign on skin were as follows, which were graded with the lowest score of 0, and the highest score of 3.9:

Healthy normal sign: 0,

Skin lesion having alopecia in a range of less than 1 cm²: 1,

Skin lesion having alopecia in a range of 1 to 2 cm²: 2,

Skin lesion having alopecia in a range of more than 2 cm²: 3, and

Skin diseases developed on ears, tail and feet: 0.3 each.

On the 34^{th} day of the transplantation, 200 μg of anti-CD137 monoclonal antibodies (3H3) isolated from immunoglobulins (Ig) as the control group (rat IgG, St. Louis, Mo.) or an ascites of the rat (provided from Dr. Mittler of Emory University) right after the cell transplantation were administered to the B10.D2→Balb/c(H-2^{d}) cGVHD mouse model. Then, the weight of the mouse model was measured from the 1^{st} to 120^{th} days of the treatment in three-day intervals, and its development signs (clinical score) were evaluated. On the 34^{th} day (the day of antibody injection) and the 58^{th} day of the transplantation, the skin on the back of the neck was checked, and then the population of the mice having ulcer was estimated in percent. After that, the skin was dissected at about 2 cm², fixed with 10% formalin, and then embedded in paraffin. The paraffin section was stained with hematoxylin & eosin, or Masson's trichrome to perform tissue analysis. The population of mice of cGVHD (%) was estimated by dividing the number of mice getting a clinical score of 0.6 or more by the total number of the mice, and multiplying the divided result by 100. A statistical significance between the test groups was tested by a Mann-Whiney method.

As a result, as shown in FIG. 1Ai, it may be confirmed from the development sign evaluation that the anti-CD137 antibody-treated mouse model recovered from the cGVHD (i of FIG. 1A). It may be also confirmed in comparison with the control group, the anti-CD137 antibody-treated mouse increased in weight (ii of FIG. 1A).

Moreover, it may be observed from the anti-CD137 antibody-treated mouse model that ulcers on all parts of the skin were treated (B and C of FIG. 1), and hairs were grown again from the skin lesions, and confirmed from the tissue analysis results that the lesions observed from the mouse model of cGVHD recovered (D of FIG. 1).

Consequently, it may be noted that cGVHD can be treated using an anti-CD137 antibody.

### Exemplary Embodiment 2

Therapeutic Effect (2) of Anti-CD137 Antibody on Developing cGVHD

Except for observation of pathological symptoms on the 58^{th} day (i.e., the 110^{th} day of the transplantation) of the antibody injection after administering the anti-CD137 antibody or Ig as the control group on the 56^{th} day of the cell transplantation to the mouse model, a therapeutic effect of the anti-CD137 antibody on cGVHD was confirmed by the same method as in Exemplary Embodiment 1.

As a result, it may be noted that the anti-CD137 antibody showed an excellent therapeutic effect on cGVHD, as when the antibody was administered on the 34^{th} day of the transplantation (A and D of FIG. 2).

Consequently, it may be noted that the anti-CD137 antibody also has an excellent therapeutic effect on severe cGVHD.

### Exemplary Embodiment 3

Therapeutic Effect of anti-CD137 Antibody on cGVHD Induced by CD4+ and CD8+ T Cells

To confirm a therapeutic effect of the anti-CD137 antibody on cGVHD induced by CD4+ and CD8+ T cells, Igs as the control group or anti-CD137 monoclonal antibodies were administered to the B10.D2→Balb/c(H-2^{d}) cGVHD mouse model obtained in Exemplary Embodiment 1 on the 30^{th} day of the cell transplantation in addition to 6×10⁶ number of un-isolated B10.D2 spleen/lymphatic gland cells, and then its development signs (clinical score) were evaluated for 30 days in three day intervals. Further, the mouse's viability was checked, the back side of the neck skin was observed on the 34^{th} day (the day of the antibody injection) and the 58^{th} day of the transplantation, the population of mice having ulcer was estimated, and the histological analysis was conducted.

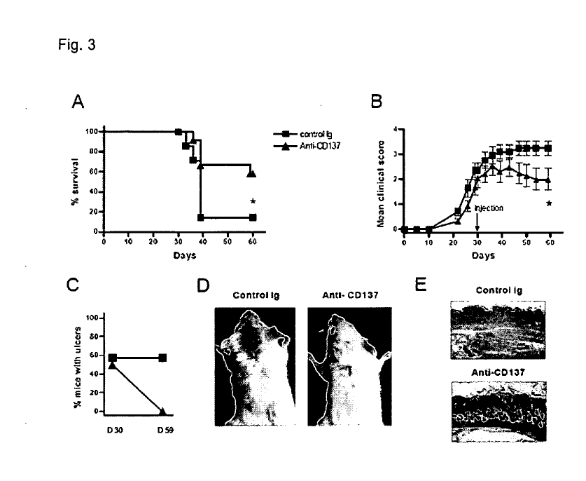

As a result, it may be confirmed that the anti-CD137 monoclonal antibody-treated mouse model was not dead (A of FIG. 3), and showed less development signs than the control group (B of FIG. 3), thereby showing an excellent effect in preventing and treating cGVHD. This therapeutic effect on cGVHD may be also confirmed from the grown hairs generated from the lesion of the mouse (D of FIG. 3) and the histological analysis results (E of FIG. 3).

Consequently, it may be noted that the anti-CD137 antibody can inhibit fatal cGVHD.

According to the present invention, a pharmaceutical composition including an anti-CD137 monoclonal antibody as an active component reduces a cytokine from a CD4+ T cell, and increases a death rate of a donor CD4+ T cell, thereby effectively preventing and treating cGVHD. Thus, this composition may be very useful for allogeneic stem cell transplantation.

Exemplary embodiments of the present invention have been disclosed herein and, although specific terms are employed, they are used and are to be interpreted in a generic and descriptive sense only and not for purpose of limitation. Accordingly, it will be understood by those of ordinary skill in the art that various changes in form and details may be made without departing from the spirit and scope of the present invention as set forth in the following claims.

What is claimed is:

1. A pharmaceutical composition for preventing and treating chronic graft-versus-host disease (cGVHD) comprising an anti-CD137 monoclonal antibody as an active component.

2. The pharmaceutical composition according to claim 1, wherein the cGVHD is caused by blood transfusion or transplantation.

3. The pharmaceutical composition according to claim 2, wherein the transplantation is one selected from the group consisting of bone marrow transplantation, stem cell transplantation and organ transplantation.
